(19) United States
(12) Patent Application Publication
(10) Pub. No.: US 2013/0008448 A1
(43) Pub. Date: Jan. 10, 2013

(54) PATIENT INTERFACE DEVICE EMPLOYING A FLOATING ADJUSTABLE ARM

(75) Inventor: Jonathan Paul Todd, Murrysville, PA (US)

(73) Assignee: KONINKLIJKE PHILIPS ELECTRONICS N.V., EINDHOVEN (NL)

(21) Appl. No.: 13/636,461

(22) PCT Filed: Feb. 24, 2011
(86) PCT No.: PCT/IB2011/050784

§ 371 Date: Sep. 21, 2012

Related U.S. Application Data
(60) Provisional application No. 61/317,429, filed on Mar. 25, 2010.

(51) Int. Cl. A61M 16/06

(52) U.S. Cl. 128/205.25

(57) ABSTRACT

A device and method of providing positive pressure support that employs a patient interface device (2, 102) having a mask (6, 106) and an adjustment arm (26, 126) coupled to the mask. The adjustment arm includes an extension member (28) extending from the mask. The mask engages the face of the patient and the adjustment arm is spaced from and not in engagement with the head of the patient. The method includes adjusting the pressure applied to the patient by the mask by adjusting a strapping force applied to the patient interface device without causing the adjustment arm to engage the head of the patient, as well as providing a flow of gas to the patient through the mask without causing the adjustment arm to engage the head of the patient.

This patent application claims the priority benefit under 35 U.S.C. §119(e) of U.S. Provisional Application No. 61/317,429 filed on Mar. 25, 2010, the contents of which are herein incorporated by reference.

The present invention relates to patient interface devices for communicating a flow of gas with an airway of a user, and in, particular, to a patient interface device that includes a floating adjustment arm, wherein adjustments to fit may be made without the adjustment arm contacting the patient's face.

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a patient interface device including a mask component on the face of a patient. The mask component may be, without limitation, a nasal mask that covers the patient's nose, a nasal cushion having nasal prongs that are received within the patient's nares, a nasal/oral mask that covers the nose and mouth, or a full face mask that covers the patient's face. The patient interface device interfaces the ventilator or pressure support device with the airway of the patient, so that a flow of breathing gas can be delivered from the pressure/flow generating device to the airway of the patient. It is known to maintain such devices on the face of a wearer by a headgear having multiple straps adapted to fit over/around the patient's head. It is also know to provide a forehead support as part of the patient interface device that is structured to provide additional support for the patient interface device by engaging the forehead of the patient

Because such patient interface devices are typically worn for an extended period of time, it is important for the headgear to maintain the mask component of the device in a tight enough seal against the patient's face without discomfort. One area where fit and comfort is often a concern is the bridge of the patient's nose, as most patient interface devices will apply a pressure to this area. If this pressure is not able to be managed effectively, either or both of a poor fit or patient discomfort will result, thereby limiting the effectiveness of the device.

The current method of providing selective adjustment for most patient interface device, such as nasal/oral masks, used in CPAP therapy includes providing the patient interface device with bottom headgear straps that are coupled to the mask, a moveable/adjustable forehead support having a moveable/adjustable forehead arm that is structured to engage the forehead of the patient, and top headgear straps that are coupled to the moveable/adjustable forehead support. While effective, this method requires a complex patient interface device having a number of movable parts.

Typically, a patient must adjust the two bottom headgear straps, the two top headgear straps, and finally the forehead arm in order to obtain a suitable fit. The philosophy behind such a system is that the forehead, which is engaged by a patient interface device component, acts as an anchor point, and by adjusting the forehead arm, the pressure at the nose bridge may be increased or decreased to account for varying facial dimensions to minimize leak while maximizing patient comfort.

In addition, conventional patient interface devices that do not employ a forehead support are adjustable through adjustment of the headgear straps alone. This results in a cumbersome and often unstable adjustment system with many adjustment points.

Accordingly, it is an object of the present invention to provide a method of providing positive pressure support to a patient that overcomes the shortcomings of conventional method. This object is achieved according to one embodiment of the present invention by providing a method of providing positive pressure support to a patient is provided that includes attaching a patient interface device to a head of the patient. The patient interface device includes a mask, first and second lower headgear straps coupled to first and second sides, respectively, of the mask, an adjustment arm coupled to the mask. The adjustment arm includes an extension member connected to and extending upwardly from a top side of the mask, and a strap frame connected to a distal end of the extension member. The mask engages the face of the patient and the adjustment arm is spaced from and not in engagement with the head of the patient. The method further includes adjusting a pressure applied to the face of the patient by the mask by adjusting a strapping force applied to the strap frame by upper headgear straps coupled to the adjustment arm without causing the adjustment arm to engage the head of the patient, and providing a flow of breathing gas to an airway of the patient through the mask without the adjustment arm engaging the head of the patient.

It is yet another object of the present invention to provide a patient interface device for use in the above-described method. The patient interface device includes a mask, and an adjustment arm coupled to the mask. The adjustment arm has an extension member operatively connected to and extending from the mask such that the adjustment arm is disposed over at least a portion of a face of such a patient. The mask and adjustment arm are configured such that when the mask engages the face of the patient, the adjustment arm remains spaced from and does not engage a head of such a patient and an air gap is provided between a head of such a patient and an entirety of the adjustment arm.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

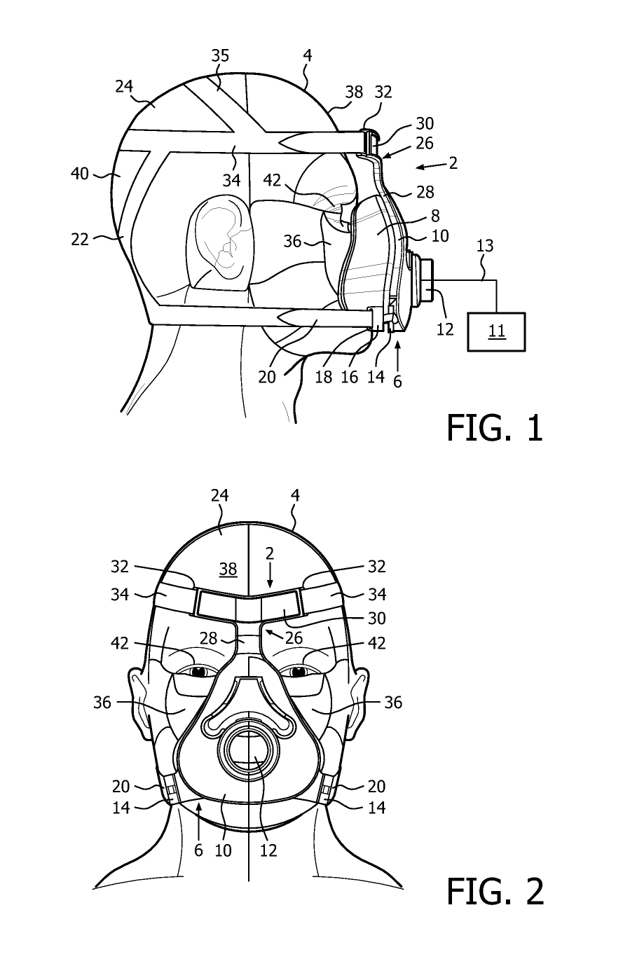

FIG. 1 is a side view of a patient interface device shown being worn by a patient according a first exemplary embodiment of the invention;

FIG. 2 is a front view of the patient interface device of FIG. 1;

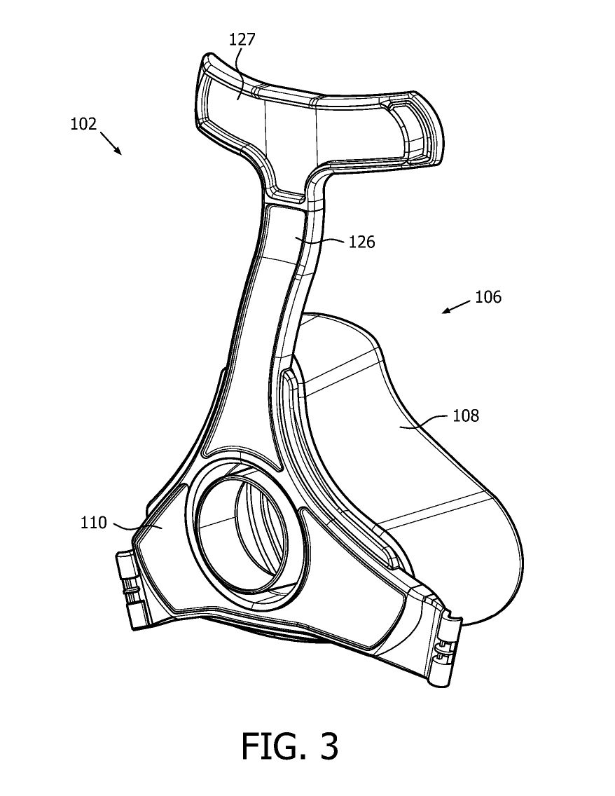

FIG. 3 is a perspective view of a second exemplary embodiment of a patient interface device according to the principles of the invention; and

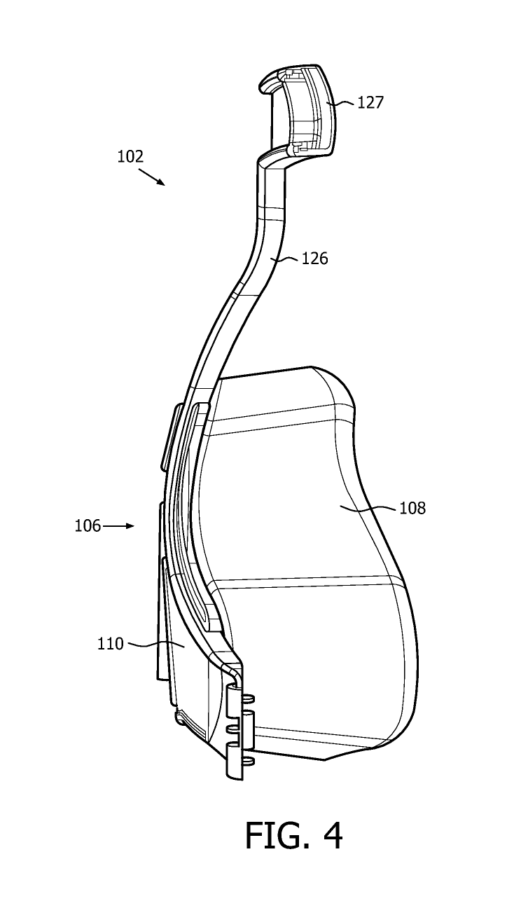

FIG. 4 is a side view of the patient interface device of FIG. 3.

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

FIGS. 1 and 2 are side and front schematic diagrams, respectively, showing patient interface device 2 being worn by patient 4 according an exemplary embodiment of the invention. Patient Interface device 2 includes mask 6, which in the exemplary embodiment is a nasal/oral mask. However, any type of mask, such as a nasal mask, a nasal pillow/cushion, or a full face mask, which facilitates the delivery of a flow of breathing gas to the airway of patient 4, may be used as mask 6 while remaining within the scope of the present invention. Mask 6 includes cushion 8 coupled to rigid or semi-rigid shell 10 (also commonly referred to as a faceplate). Shell 10 may be made of a material such as, without limitation, polycarbonate or reinforced silicone.

In the exemplary embodiment, cushion 8 is made of a soft, flexible material, such as, without limitation, silicone, an appropriately soft thermoplastic elastomer, a closed cell foam, gel, or any combination of such materials. Opening 12 in shell 10 is structured to be coupled to a coupling connector (not shown), such as an elbow connector, which in turn is coupled to a pressure generating device 11 via a suitable delivery conduit 13. Opening 12 allows the flow of breathing gas generated by the pressure generating device to be communicated to an interior space defined by shell 10 and cushion 8, and then to the airway of patient 4. Pressure generating device 11 may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices.

Shell 10 includes first and second slots 14 each structured to receive and hold a catch of clip element 16. In the exemplary embodiment, slots 14 and clip elements 16 are structured in the form of a ball and socket configuration, although other configurations are also possible. Each clip element 16 also includes loop 18 for receiving a respective lower headgear strap 20 of headgear component 22 used to secure patient interface device 2 to head 24 of patient 4. The length of each lower headgear strap 20 is selectively adjustable by way of a hook and loop fastening system, such as VELCRO®. In particular, the exterior of each lower headgear strap 20 includes a loop fastener portion, and a corresponding hook fastener portion is provided on the exterior of the end portion of the lower headgear strap 20. Thus, each lower headgear strap 20 may be threaded through a respective loop 18 and then bent back on itself in order to adhere the hook fastener portion to the loop fastener portion at a selected location. Other selectively adjustable fastening arrangements are also possible.

Patient interface device 2 further includes a floating adjustment arm 26 coupled to shell 10. More specifically, adjustment arm 26 includes rigid or semi-rigid extension member 28 that is rigidly (i.e., non-moveably) coupled to and extends upwardly from shell 10. Strap frame 30 is rigidly (i.e., non-moveably) coupled to the distal end of extension member 28. Adjustment arm 26 may be made of a material such as, without limitation, polycarbonate or reinforced silicone. Strap frame 30 includes loops 32 provided at opposite ends thereof. Each loop 32 is structured to receive a respective upper headgear strap 34 of headgear component 22. As seen in FIG. 1, each upper headgear strap 34 extends from strap frame 30 along a respective side of head 24 of patient 4 toward rear 40 of head 24.

In the exemplary embodiment, strap frame 30 is positioned above eyes 42 of patient 4 and thus upper headgear straps 34 extend at a level above and out of the way of eyes 42. The length of each upper headgear strap 34 is selectively adjustable by way of a hook and loop fastening system as described above. In addition, in the exemplary embodiment, headgear component 22 includes crown strap portion 35 to account for vertical forces resulting from use of patient interface device 2.

It can be appreciated that extension member 28 and/or strap frame 30 can have a variety of different configurations, shapes, and sized. For example, strap frame 30 can be omitted entirely so that the headgear straps attach directly to extension member 28. The present invention also contemplates that extension member 28 and strap frame 30 can be made from a variety of materials or combinations of materials. Moreover, extension member 28 and strap frame 30 can be formed as a unitary structure or can be separate structures that can be selectively detached from one another. Also, the present invention contemplates that more than one extension member can be provided. For example, two extension members can extend from the shell. A strap frame provided on each or can be omitted.

As seen in FIGS. 1 and 2, when patient interface device 2 is in use and worn by patient 4, cushion 8 of mask 6 engages face 36 of patient 4 around the mouth and bridge of the nose. However, adjustment arm 26 does not—during use of patient interface device 2—contact head 24, and in particular forehead region 38, of patient 4. As seen in FIG. 1, adjustment arm 26 remains spaced from head 24 of patient 4 during use. Patient 4 may selectively adjust the nose bridge pressure applied by mask 6 simply by adjusting the length of, and thus the strapping force applied by, upper headgear straps 34. When this is done, adjustment arm 26 acts as a cantilever to transfer the force and the adjustment of upper headgear straps 34 to the bridge of the nose of patient 4. This cantilever effect allows direct adjustment of the upper portion of mask 6 at the bridge of the nose of patient 4 without headgear straps interfering with eyes 42 of patient 4 and without adjustment arm 26 engaging head 24 of patient 4. In contrast to the prior, there is also no need to adjust any part of adjustment arm 26, as it is a rigid or semi-rigid component with no moving parts.

However, the present invention does contemplate allowing for adjustment of the effective length of adjustment arm 26, so long as the adjustment arm does not contact the user. Adjusting the length can be accomplished, for example, by providing a sliding coupling to the mask shell and/or a telescoping feature on the adjustment arm.

In an alternative embodiment, each upper headgear strap 34 may be made of a material having sufficient elastic properties to provide for automatic adjustment of the pressure applied to face 36 of patient 4 by mask 6 while also providing for an effective seal to be obtained between cushion 8 of mask 6 and face 36 of patient 4 without the need to manually adjust the length of the upper headgear straps 34.

Patient interface device 2 as just described is advantageous in that it provides a simple design with fewer (and non-moving) parts that allows for simpler adjustment of nose bridge pressure by patient 4 or a therapist. The simple design translates into lower manufacturing costs. In addition, with fewer (and non-moving) parts, the chances of breakage are reduced and quality is thus increased. Also, less adjustment points (i.e., no need to adjust adjustment arm 26) helps patient 4 or a therapist to achieve a suitable seal between mask 6 and face 36 of patient 4 more quickly, which will promote further therapy and product use.

FIGS. 3 and 4 illustrate a patient interface device 102 according to an alternative embodiment of the present invention. Patient interface device 102 includes a mask 106 having a cushion 108 and a shell 110. Cushion 108 is coupled to shell 110 using any suitable configuration. In an exemplary embodiment, however, cushion 108 is removable from shell 110 to that the cushion can be replaced. In addition, a floating adjustment arm 126 is operatively coupled to shell 110 and headgear attaching elements are provided on the shell and a distal end 127 of adjustment arm 126.

It can be appreciated that is many respects, patient interface device 102 is generally similar to patient interface device 2 of FIGS. 1 and 2. For example, when worn by the user, adjustment arm does not contact the user. Patient interface device 102 is provided to show alternative exemplary configurations for the mask and the floating adjustment arm. For example, in this embodiment, mask 106 is a nasal mask that is sized and configured to fit over only the nose of the user.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A method of providing positive pressure support to a patient, comprising:
(i) attaching a patient interface device to a head of the patient, the patient interface device comprising:
(a) a cushion,
(b) a frame member including a shell having the cushion coupled thereto an adjustment arm coupled to the shell, the adjustment arm having an extension member rigidly and non-moveably connected to and extending upwardly from a top side of the shell and a strap frame rigidly and non-moveably connected to a distal end of the extension member, the shell and strap frame forming a T-shaped member, the shell, the extension member and the strap frame being a unitary structure,
(c) first and second lower headgear straps operatively coupled to first and second sides, respectively, of the shell, and
(d) first and second upper headgear straps operatively coupled to first and second opposite sides, respectively, of the strap frame, each of the first and second headgears straps extending along a respective side of the head of the patient, wherein the cushion engages the face of the patient and the adjustment arm is spaced from and not in engagement with the head of the patient;
(ii) adjusting a pressure applied to the face of the patient by the shell and the cushion by adjusting an upper strapping force applied to the adjustment member by the upper headgear straps, a lower strapping force applied to the mask by the lower headgear straps, or by adjusting both the upper and the lower headgear straps without causing the adjustment arm to engage the head of the patient; and
(iii) providing a flow of breathing gas to an airway of the patient through the shell and the cushion without the adjustment arm engaging the head of the patient.

2. The method of claim 1, wherein the first and the second upper headgear straps are adjustable, and wherein the strapping force applied to the adjustment arm by the upper headgear straps is selectively adjusted by adjusting a length of the upper headgear straps.

3. The method of claim 1, wherein the upper headgear straps are made of an elastic material, and wherein the strapping force applied to the adjustment arm by the upper headgear straps is adjusted as a result a stretching of the upper headgear straps in response to the attaching of the patient interface device to the head of the patient.

4. The method according to claim 1, wherein the adjustment arm functions as a cantilever to cause a top portion of the cushion to deliver a nose bridge force to a bridge of the nose of the patient in response to the strapping force.

5. The method of claim 1, wherein the upper headgear straps are positioned above a level of the eyes of the patient.

6. The method of claim 1, wherein the patient interface device is one of a nasal/oral mask, a nasal mask, and a nasal cushion.

7. (canceled)

8. (canceled)

9. The method of claim 1, wherein the adjustment arm and the shell are made of a rigid or semi-rigid material.

10. The method of claim 9, wherein the adjustment arm has no moving parts.

11. (canceled)

12. A patient interface device adapted to be coupled to a head of a patient, the patient interface device comprising:
a cushion,
a frame member including a shell having the cushion coupled thereto and an adjustment arm coupled to the shell, the adjustment arm having an extension member rigidly and non-moveably connected to and extending from a top of the shell and a strap frame rigidly and non-moveably connected to a distal end of the extension member, the shell and strap frame forming a T-shaped member, the shell, the extension member and the strap frame being a unitary structure,
first and second lower headgear straps operatively coupled to first and second sides, respectively, of the shell, and
first and second upper headgear straps operatively coupled to first and second opposite ends, respectively, of the strap frame, wherein the cushion, the first and second lower headgear straps, the first and second upper headgear straps and the frame member are configured such that when the cushion engages the face of the patient, the adjustment arm remains spaced from and does not engage a head of such a patient and an air gap is provided between a head of such a patient and an entirety of the adjustment arm.

13. (canceled)

14. (canceled)

15. (canceled)
